(19) United States
(12) Patent Application Publication
(10) Pub. No.: US 2024/0054164 A1
(43) Pub. Date: Feb. 15, 2024

(54) A DATA EXTRACTION SYSTEM

(71) Applicant: SEROTONIN LABS INDIA PRIVATE LIMITED, BENGALURU URBAN KARNATAKA BANGALORE (IN)

(72) Inventors: Shefali SABHARANJAK, Karnataka Bangalore (IN); Rajesh Tanamala Srinivas REDDY, Koramangala Bangalore (IN); Shobini Kaveriappa APPANDERANDA, San Francisco, CA (US); Deeksha SHARMA, Shivarama Karanth Nagar Bangalore (IN); Roopa SHANKARANARAYANA, Nagar Bengaluru (IN); Sindhulakshmi Dhanesh KURUP, Tiruvalla Kerala (IN)

(73) Assignee: SEROTONIN LABS INDIA PRIVATE LIMITED, BENGALURU URBAN KARNATAKA BANGALORE (IN)

(21) Appl. No.: 18/258,304

(22) PCT Filed: Dec. 17, 2021
(86) PCT No.: PCT/IN2021/051184

§ 371 Date: Jun. 19, 2023

(30) Foreign Application Priority Data
Dec. 17, 2020 (IN) 202041055074

(51) Int. Cl. G06F 16/93; G06F 16/38; G06F 16/338; G06F 40/30; G16H 10/20; G06F 3/0482

(52) U.S. Cl. CPC G06F 16/93; G06F 16/38; G06F 16/338; G06F 40/30; G16H 10/20; G06F 3/0482

(57) ABSTRACT

Example approaches for associating relevant tags for facilitating searching and extracting relevant medical documents, are described. In an example, a document characteristic field of the documents extracted from a database repository are tagged with tag values to obtain a tagged document. Thereafter, the tagged document is stored in the database repository. To search the tagged document, a target value for the document characteristic field is obtained and compared with the tag values of the tagged documents. Based on the result of comparison, a list of tagged documents is extracted from the database repository and displayed for user's review.

### TECHNICAL FIELD

The present subject matter relates to data extraction, and in particular to tagging a document with relevant tags for facilitating searching and extraction of necessary and relevant information from the document.

### BACKGROUND

The search for linkages, associations and correlations between different health parameters, conditions and goals for increasingly complex medical problems has become a key focus for medical and health/wellness practitioners (collectively referred to as wellness service providers) to address health related issues. Some solutions to these problems may be found by looking for knowledge using different information sources or medical documents, such as document related to clinical trials, medical research studies, etc. Clinical trials provide an evaluation of the merits of using one or more treatment options for given disease or health condition of interest. As knowing the human limitation, it is almost impossible for a single person to go through all of available documents and make a cogent conclusion out of it. To overcome this problem, various methods and techniques have been developed, such as keyword-based search and content recognition search to segregate meaningful medical document from the vast available dataset. However, these conventional methods and techniques provides unstructured or non-categorized dataset of documents having large number of documents with overlapping fields. Further, these methods do not provide targeted document for which a user is looking for. Also these methods cannot scale up to handle a large volume of documents that have disparate formats and sources.

### BRIEF DESCRIPTION OF DRAWINGS

The detailed description is provided with reference to the accompanying figures, wherein:

FIG. 1 illustrates a data communication environment with a data extraction system, in accordance with exemplary implementation of the present subject matter;

FIG. 2 illustrates an exemplary view of document extracted from the database repository displayed in a listed manner, in accordance with one implementation of the present subject matter;

FIG. 3 illustrates an exemplary view of extracted documents initially searched based on a document related attributes for individually selecting the document, in accordance with one implementation of the present subject matter; and

FIG. 4A-4D illustrates an exemplary view of an individually selected document with corresponding document characteristic fields for tagging, in accordance with one implementation of the present subject matter;

FIG. 5 illustrates an exemplary view of a search screen facilitating searching of the tagged documents by entering target values in corresponding document characteristic fields, in accordance with one implementation of the present subject matter;

FIG. 6 illustrates a flowchart depicting exemplary method for associating tag values with a document to obtain a tagged document, in accordance with one implementation of the present subject matter; and

FIG. 7 illustrates a flowchart depicting exemplary method for

searching and extracting a document using associated tag values, in accordance with one implementation of the present subject matter.

It may be noted that throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Clinical trials may involve a variety of scientific studies that examine and evaluate the safety and efficacy of any new invented drugs, tests, or devices through various interventions on living organism, such as a human, as a subject. The clinical findings obtained from these clinical trials are considered to be the most relevant data in the modern era for evidence-based health management strategies. As would be understood, a vast variety of clinical trials are conducted across the globe each having results or finding related to different medical conditions, effects or problems, and with different set of criteria in relation to the drug being tested. In general, all the clinical trials are classified in two broader categories, such as intervention-based studies and observational studies. Some of the examples of criteria on which these clinical trials are being conducting are, but may not be limited to, target subject, study design, study size, ingredient used, health benefit, health conditions, etc. As mentioned previously, different clinical trial data have different set of results and different set of preconditions with which they are dealing, so it will pose great challenge for a wellness service provider to find the right source of information from vast available information source.

Generally, these trials are conducted by individual wellness service providers of different organisations. Once these clinical trials are conducted with proper protocol, the various outcome and findings may be recorded in the form of documentation, papers or similar publication including findings segregated in different sections, namely, abstract, summary, introduction, and conclusion, which may help drug developers and other practitioners in the medical field. Such documents or publications may be available across multiple data sources and/or repositories which in turn may be accessible through computer implemented networks. Using such search engines, investigators of different medical fields may access these trial data for secondary uses.

Various conventional approaches provide a variety of techniques for searching documents related to clinical trials. Examples of such conventional approach involves keyword-based selection process and content recognition technique in order to achieve or find relevant results. Considering the large number of documentations or publications (collectively referred to as documents), it is not possible to selectively determine and retrieve documents with fine granularity, particularly in relation to other pertinent categories which may be related to the health situation which may be under consideration as part of clinical trials. Furthermore, the documents may be available in a non-categorised, unstructured form which further creates challenges in finding and retrieving relevant information pertaining to one or more clinical trial specific information. In light of above drawbacks there is a clear need for a system which can perform categorization of the documents based on their respective trial attributes or fields and further this categorization may help in searching and retrieval of the document from a large database.

Examples of data extraction systems for associating relevant tags for facilitating searching and extracting relevant medical documents, are described. In one example, the data extraction system is communicatively coupled over a network with a database repository, which may include a large number of documents related to clinical trials or medical research studies. The database repository contains several documents and/or publications related to clinical trials gathered from a variety of efforts which range from intervention-based trials to observational trials. Besides intervention-based trials or observational trials, clinical trials may also be based on a variety of other factors which include, but are not limited to, types of drugs or pharmaceutical agents, compositions, combinations of drugs, herbs, lifestyle modifications, and diet. It may be noted that the present list of factors is only indictive, and other factors, based on which clinical trials are conducted, may also be considered without deviating from the scope of the present subject matter. Further, such documents or publications may not be available at a single repository but may be available across multiple repositories. The repository in turn may be a single computer accessible resource or may be implemented as a combination of multiple computer accessible resources, without deviating from the scope of the present subject matter.

As may be understood, clinical trial is a process which involves conducting and recording information pertaining to different aspects, such as target group, health condition, health categories, co-supplement, etc. To facilitate searching and retrieval of such documents, certain document characteristics fields are being associated or tagged with tag values based on the conceptual understanding of the document and accordingly searched using these tag values. The relevant tag values are associated with each of these trial specific fields based on the analysis of the document. It may be noted that the associated tag values may have different types of values for different fields. For example, age, study size or dosage related fields have numerical tag value and gender, ingredient or health condition related fields may have alphabetical word-based tag value. These tag values are associated with the retrieved documents based on the content of the retrieved document or inputs provided by the user after analysing or reading the retrieved document.

It may be noted that, after tagging, each document is stored in the database repository allowing the documents to be searched by specifically providing values for the tag values. In an example, the tagged documents are stored in the similar or different database repository, which may be utilised for searching and extraction of such documents based on their respective tag values associated with document specific fields.

In operation, during tagging, initially the data extraction system may be populated by retrieving or extracting documents from a data base repository. It may be noted that the documents may be extracted or retrieved based on a search query provided, either by a user based on their personal preferences, or by a medical practitioner based on its requirement, without limiting the scope of the present subject matter. The data extraction system, on receiving the search query, may extract a document identification information from the search query. In an example, the document identification information comprises information regarding type of document, e.g., either clinical trials, medical research studies or other type of medical documents, type of section of the document, e.g., either abstract, introduction, summary, conclusion, or other type of sections, and species on which the document is related, e.g., either human, animal, mammals, reptiles, or other type of species.

Once the documents are extracted, an individual document is selected for tagging. In an example, a user input is received from the user to select one of the documents from the extracted documents. Once selected, the data extraction system may display the selected document with a plurality of document characteristic fields for receiving a value for each field. Examples of such document characteristic fields include, but are not limited to, health categories, health condition, co-supplement used, co-morbidities, target group, ethnicity, genotype, formulation, dosage, frequency, duration, study type, clinical trial rating system, study size, age group, gender, effects, efficacy ratings, adverse effects, and negative biomarker effect. In an example, the document characteristic fields of each document are a set of predefined fields depicting conceptual understanding of content present in that document. It may be noted that the present set of examples of document characteristic fields is only indicative. Other examples may also be provided without limiting the scope of the present subject matter.

Returning to the foregoing example, once the selected document with corresponding document characteristic fields is displayed, a tag value for each of the document characteristic field is provided by the user based on the content analysis of the selected document. Thereafter, the tag values are associated with corresponding document characteristic fields. Tag values for each of the document characteristic fields are entered by the user by selecting one of the values provided as a drop-down menu or may be defined through user input. Once all values corresponding to each document characteristic fields are obtained and associated, a tagged document is including tag values for each of the document characteristic fields is stored in the database repository. In an example, the tagged document is stored in the database repository. It may be noted that, tag values represent corresponding values for each document characteristic fields associated based on the content-based analysis of the selected document by the user. In an example, the document characteristic fields further comprise sub-fields which correspond to a set of tag values from amongst the plurality of the tag value entered by the user for corresponding document characteristic field.

As may be appreciated, once the database repository is filled with large number of such tagged documents, a content specific tag-based search may be performed on receiving a search request from a user. For example, a search request specifying certain target values of document characteristic fields may be received from the user. The data extraction system may then compare the target values with the tag values of corresponding document characteristic fields of each tagged document in the data repository. Based on the result of comparison, a list of tagged documents is retrieved from the data repository and displayed for user's review. In an example, the tagged documents in the displayed list are prioritized based on the number of successful matching of target values with corresponding tag values. For example, documents having high number of correct matching are displayed on higher position in the list and documents having low number of matching are displayed on lower position in the list.

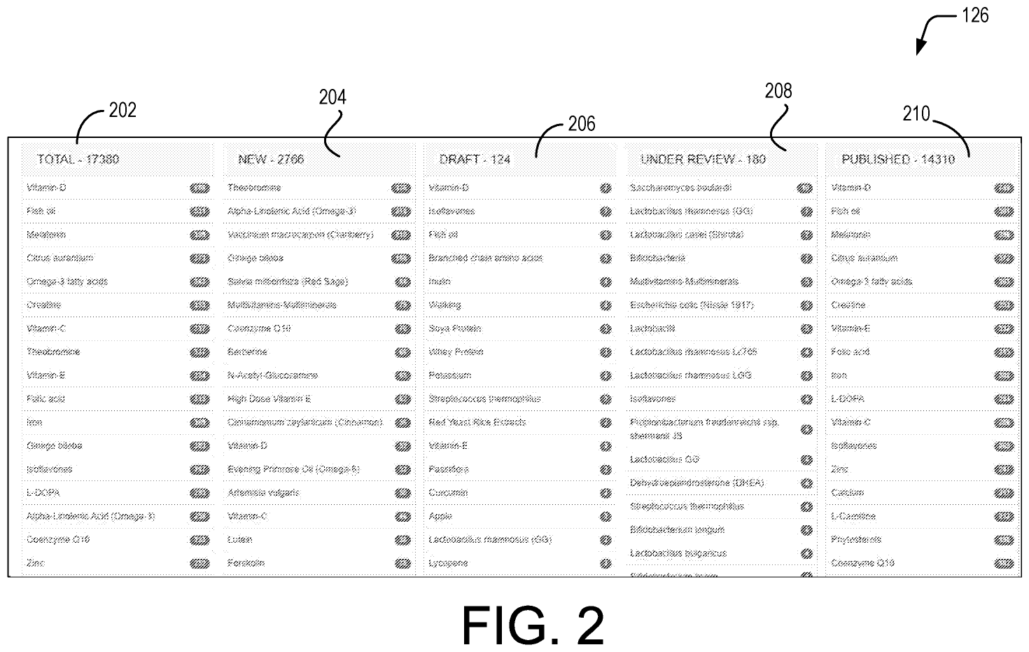

The manner in which the example data extraction system is used for tagging and searching the documents is further explained in detail with respect to FIGS. 2-7. It is pertinent to note that the exemplified approaches have been explained with reference to documents and publications derived from a specific type of clinical trials. Such approaches may be followed for any type of clinical trial without limiting the scope of the subject matter for which protection is sought. It is to be noted that drawings of the present subject matter shown here are for illustrative purposes and are not to be construed as limiting the scope of the subject matter claimed.

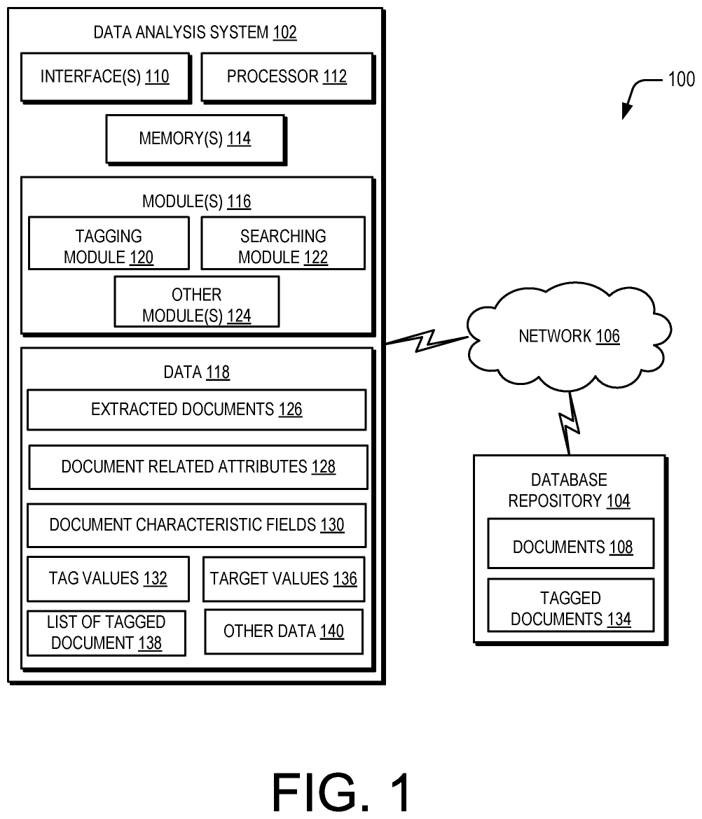

FIG. 1 illustrates a data communication environment 100, comprising a data extraction system 102. The data extraction system 102 (referred to as a system 102) performs tagging of a document for facilitating searching of the document, as per an example of the present subject matter. The system 102, in an example, may relate to any system capable of receiving user's inputs, processing it, and correspondingly provide output based on the received user's inputs.

The system 102 may be coupled to a database repository 104 over a communication network 106. The database repository 104 may be implemented as any hardware or software-based repository which may be able to store data. In one example, as depicted in FIG. 1, the database repository 104 may be connected with the system 102 over the communication network 106. In another example, the database repository 104 may be present within the system 102.

In yet another example, the database repository 104 may be implemented over a centralized computing server, and may be in communication with the system 102 over the communication network 106. The database repository 104 includes a plurality of document(s) 108, such as various clinical trial documents, medical research studies, etc. In one example, the documents 108 may relate to one or more types of clinical trials without limiting the scope of the present subject matter. The database repository 104 in turn may be implemented using a single storage resource (e.g., a disk drive, tape drive, etc.), or may be implemented as a combination of communicatively linked storage resources (e.g., in the case of Infrastructure-as-a-Service), without deviating from the scope of the present subject matter.

The system 102 may include interface(s) 110, processor 112, a memory 114, and an. The interface(s) 110 may allow the connection or coupling of the system 102 with one or more other devices, through a wired (e.g., Local Area Network, i.e., LAN) connection or through a wireless connection (e.g., Bluetooth®, WiFi). The interface(s) 110 may also enable intercommunication between different logical as well as hardware components of the system 102. The processor 112 may be implemented as microprocessors, microcomputers, microcontrollers, digital signal processors, central processing units, state machines, logic circuitries, and/or other devices that manipulate signals based on operational instructions.

The memory 114 may be a computer-readable medium, examples of which include volatile memory (e.g., RAM), and/or non-volatile memory (e.g., Erasable Programmable read-only memory, i.e., EPROM, flash memory, etc.). The memory 114 may be an external memory, or internal memory, such as a flash drive, a compact disk drive, an external hard disk drive, or the like. The memory 114 may further include data which either may be utilized or generated during the operation of the system 102.

The system 102 may further include module(s) 116 and data 118. The module(s) 116 may be implemented as a combination of hardware and programming logic (e.g., programmable instructions) to implement one or more functionalities of the module(s) 116. In one example, the module(s) 116 may include a tagging module 120 for associating a tag value to one or more document characteristic fields corresponding to the plurality of documents 108 retrieved from the database repository 104 and a searching module 122 for searching a target document amongst the tagged documents based on the associated tag values. The module(s) 116 may further include other module 124. The other module 124 may implement functionalities that supplement applications or functions performed by the system 102 and module(s) 116.

The data 118 on the other hand includes extracted documents 126, document related attributes 128, document characteristic fields 130, tag values 132, target values 136, list of tagged document 138, and other data 140. Further, the other data 140, amongst other things, may serve as a repository for storing data that is processed, or received, or generated as a result of the execution of one or more modules in the module(s) 116.

In examples described herein, such combinations of hardware and programming may be implemented in a number of different ways. For example, the programming for the module(s) 116 may be processor executable instructions stored on a non-transitory machine-readable storage medium and the hardware for the module(s) 116 may include a processing resource (e.g., one or more processors), to execute such instructions. In the present examples, the machine-readable storage medium may store instructions that, when executed by the processing resource, implement module(s) 116 or their associated functionalities.

In operation, a user, such as a data scientist or any other general user, may initiate the process of tagging by retrieving or extracting documents 108 from the database repository 104 over the communication network 106 based on a search query. In an example, the database repository 104 corresponds to any open source database which contains large number of documents 108 related to the clinical trial studies. The documents 108 retrieved or extracted from the database repository 104 may be stored as extracted documents 126 in the system 102. In one example, FIG. 2 depicts the manner in which document extracted from the database repository displayed in a listed manner. It may be note that the extracted documents 126 are segregated and displayed in different columns, such as total documents, new documents, draft documents, under review documents, and published documents. In one example, the manner in which extracted documents 126 displayed in different columns is depicted in FIG. 2.

Continuing with the present example, before selecting individual document for tagging, the user may select or retrieve the documents based on document related attributes 128 to specifically search relevant documents for tagging. Examples of such document related attributes 128 include, but may not be limited to, supplementary intervention used in the document, document repository ID, health benefit and health condition disclosed in the document.

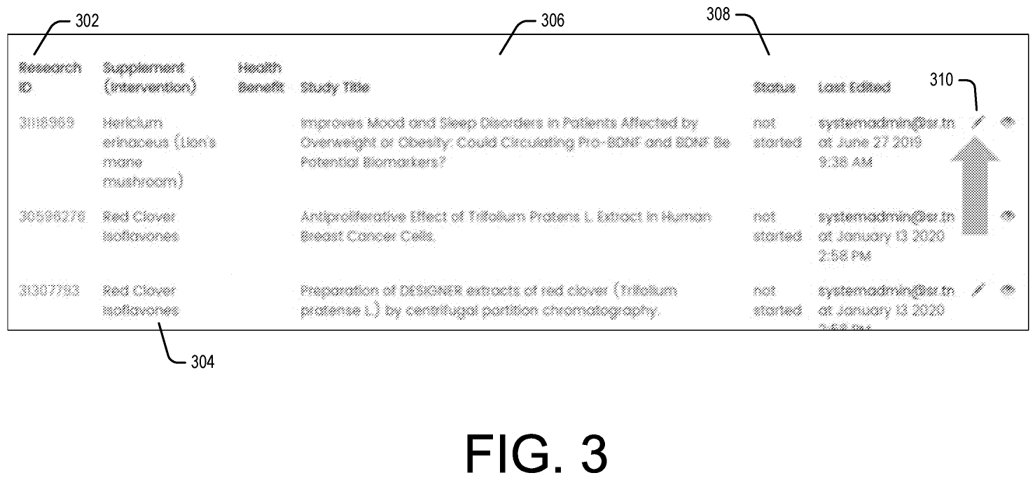

Once the extracted documents 126 are populated and segregated based on the document related attributes 128, the user may select an individual document for tagging. In one example, FIG. 3 depicts the manner in which the extracted documents initially searched based on a document related attributes for individually selecting the document. On selecting individual document, the system 102 displays a plurality of document characteristics fields 130 for associating a tag value(s) 132 with each document characteristic field. Examples of such document characteristic fields 130 include, but are not limited to, health categories, health condition, intervention, co-supplement used, co-morbidities, target group, ethnicity, genotype, formulation, dosage, frequency, duration, study type, clinical trial rating system, study size, age group, gender, effects, efficacy ratings, adverse effects, negative biomarker effect, etc.

Indicative examples of the document characteristic fields 130 and corresponding possible tag value(s) 132 are listed below, as per one implementation, in Table A. The explanation hereby is for purposes of explaining certain examples of the present subject matter. The explanations provided below are not to construed as limiting the scope of the subject matter in anyway.

TABLE A

| | |
|---|---|
| 1. | Health Categories: These are areas of human health that have been |
| | created broadly based on human anatomy and also on major health |
| | functions e.g. Liver health, Brain health, Mental health etc. |
| 2. | Health Conditions: These segregations are sub-sections of a health |
| | category. E.g. Depression is a Health condition under Health |
| | Category Mental Health. Health conditions essentially are states of |
| | health that are specific and distinct from other states of health that |
| | fall under the broad umbrella of a health category. These |
| | distinctions can be made based on functional, biochemical and |
| | structural differences with other health conditions. |
| 3. | Intervention: An intervention can be either a diet pattern, a lifestyle |
| | choice or a dietary supplement. These may be present singly or in |
| | combinations. Drugs included in the trials may also be recorded as |
| | interventions in a separate field |
| 4. | Co-morbidities: Some health conditions like chronic health |
| | problems may exist in individuals. In some studies, another health |
| | issue may be studied in the background of a chronic health |
| | problem. |
| | E.g. Muscle strength development may be studied in people with |
| | Diabetes that is not dependent on Insulin. Hence, the presence of |
| | diabetes and the type of diabetes is recorded as a ‘Co-Morbidity’. |
| 5. | Co-Supplements: Two or more dietary supplements may be utilized |
| | as a combination supplement. In this case, one supplement is |
| | nominated as the main ‘Intervention’ in the study and the other |
| | ones is termed as the ‘Co-Supplement (s)’. |
| 6. | Target Group: Target groups are identifier tags that are independent |
| | of the health status of the individual. Eg. Athletes, Healthy Adults |
| 7. | Ethnicity: Ethnicity is racial information about the participants of a |
| | clinical trial. This is mentioned as per standard identification tags |
| | like Asian, Caucasian, African, etc. |
| 8. | Genotype: Genotype or genetic profiles are identifiers based on the |
| | presence of specific variants of human genes. This information is |
| | captured in the Genotype field to identify health effects ascribed to |
| | certain genetic backgrounds. |
| 9. | Formulation: This includes the brand name of the formulation of |
| | the intervention and the proportions of the ingredients included. |
| 10. | Dosage: The dosages of the interventions administered are |
| | recorded in this format: Name, dosage numerical value and dosage |
| | units. Multiple dosages are expressed in separate fields in the same |
| | format. |
| 11. | Frequency: This field captures the number of times an intervention |
| | is administered to the participants of a trial. The Frequency is |
| | mentioned as a numerical value and in units such as twice a day, |
| | thrice a day etc. |
| 12. | Duration: Duration of a study is entered into a separate field. The |
| | duration is split into study duration and Duration of intervention |
| | administration fields. Study duration is recorded in the format of |
| | numerical format (numbers) and units (weeks, days, months, years, |
| | as applicable). Duration of the follow-up period or washout |
| | period is also recorded in the duration fields. |
| 13. | Study Type: Clinical trials are conducted in several formats and |
| | over the past two decades there have been standards developed |
| | for the conduction of these clinical trials. Industry standards like |
| | Double-blind, Triple-Blind, Randomized, Placebo-controlled |
| | and others are used to label and classify clinical trials. In our |
| | system, these labels are applied as per the descriptions provided |
| | in the clinical trial publication. |
| 14. | Clinical trial Rating System: A numerical weightage is assigned to |
| | each label (described in the previous field) and a clinical trial rating |
| | system has been created. Essentially the sum of the numerical |
| | values of the labels is assigned as a score to the clinical trial. This |
| | system is used to judge the merit of the clinical trial and assign |
| | appropriate weightage to the results described therein. |
| 15. | Study Size: The number of participants in a clinical trial is captured |
| | as the study size. A numerical value is assigned to this field. |
| 16. | Age and Age group: The ages of the participants are captured in |
| | two fields: Age groups as per a present classification and actual age |
| | ranges in numerical values as described in the papers. |
| | Young Adult (19- 40 years) |
| | Middle- Aged (40-65 years) |
| | Elderly (65- 80 years) |
| | Geriatric (80 years and above) |
| | Adult (19-100, if a better classification is not provided) |
| 17. | Gender: Gender of the participants is recorded as Men or women |
| | or both, as described in the trial publication |
| 18. | Effects: The effects of the intervention are captured verbatim from |
| | the paper and classified as: |
| | Improvement or Increase in health parameters, biomarkers or |
| | clinical status |
| | Reduction or Decrease in health parameters, biomarkers or clinical |
| | status |
| | No effect in health parameters, biomarkers or clinical status |
| | There is no limitation on the number of effects that can be captured |
| | from a published trial document |
| 19. | Effect class: Most clinical trial effects are captured in scientific |
| | language to retain the original discovery intact. However, similar |
| | effects can also be described in an effect class like ‘Provides relief |
| | from migraines’ or ‘Reduces systemic inflammation’. These effect |
| | classes are important for consumerisation as well as grouping of |
| | the changes in biochemical markers in buckets that capture the |
| | physiological mechanism of the effects of the interventions. |
| 20. | Efficacy Ratings: A significance rating is assigned to each effect |
| | captured from the clinical trials, separately. Most biological results |
| | are ratified by using statistical tests applied to datasets. A large |
| | number of statistical tests assign significance cut offs in the form of |
| | a P value (or other statistical test coefficients) and other qualifiers |
| | like data intervals. The P values described in clinical trial |
| | publications are used to create an efficacy rating system of 0 = no |
| | change from controls/ placebo, 1 = noticeable change but not |
| | significantly different from the controls/placebo and 2 = important |
| | effect, statistically significantly different from control/placebo. Plus |
| | (+) and minus (−) signs are used to indicate an increase or decrease |
| | in the trend of change of the parameters assessed. |
| | If change is statistically significant (p < 0.05) or >20% over |
| | placebo then “+2 or −2”; |
| | P values not significant but data indicates a trend of change in |
| | supplement Vs placebo then “+1 or −1”; |
| | No effect of intervention- then “0”, |
| | For reporting safety/tolerability, as no P value is given, if there is |
| | no side effect and good safety and tolerability mentioned, +1 |
| | value is given. |
| 21. | Negative Biomarker Effect: If a biomarker is affected in a way that |
| | is physiologically disadvantageous, the negative biomarker tag is |
| | used to mark this effect and that publication. These results and |
| | papers are excluded from the algorithm used to personalise and |
| | choose profile-specific interventions. The findings in these papers |
| | are evaluated separately before inclusion into the intervention |
| | choice algorithm. |
| 22. | Adverse Effects: Undesirable side effects of an intervention, if |
| | declared in the publication, are captured in this field. |
| 23. | Comments and Reasons for Invalidation: If a study is deemed as |
| | not fit for inclusion in the data set of interest, then the reasons for |
| | invalidation are mentioned in this field. |

It may be noted that additional document characteristic fields 132 may be added for further categorisation of the selected document. In an example, the tag value(s) 132 for each document characteristic fields 130 listed above are provided by a user, such as data scientist, after analysing the content of the selected document and these tag value(s) 132 are then tagged with corresponding document characteristic fields 130. In an example, the document characteristic fields 130 further comprise sub-fields which correspond to a set of tag values from amongst the plurality of the tag value entered by the user for corresponding document characteristic fields 130.

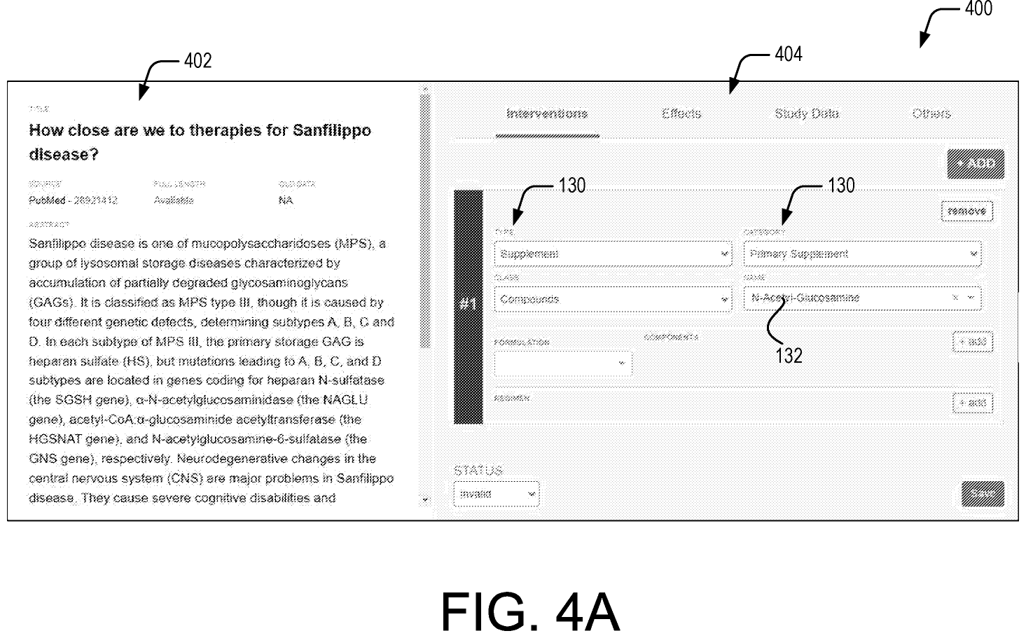

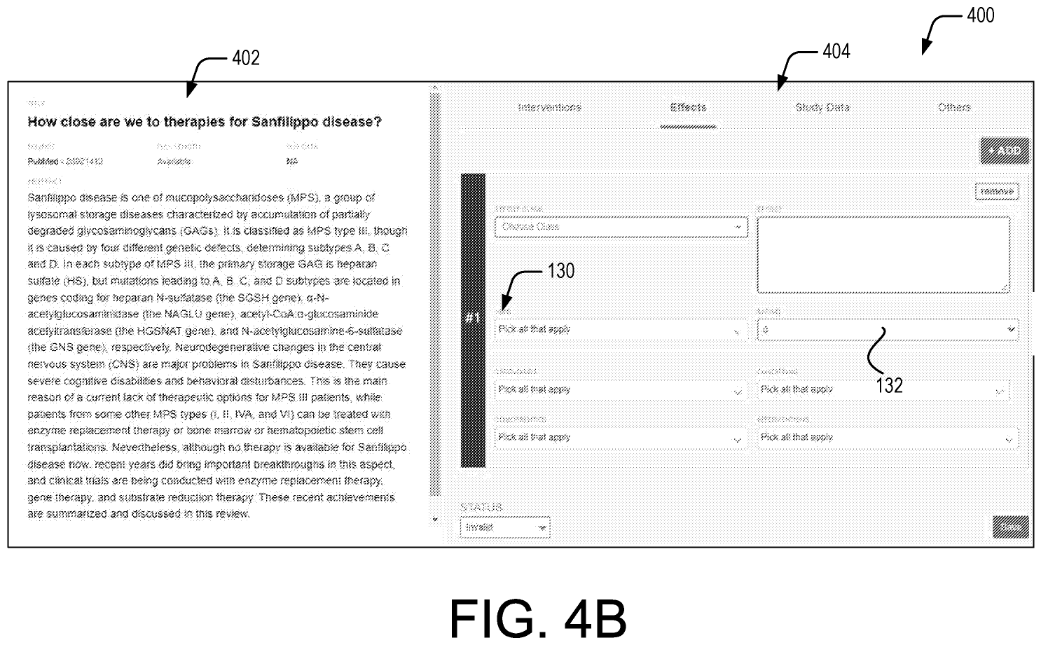

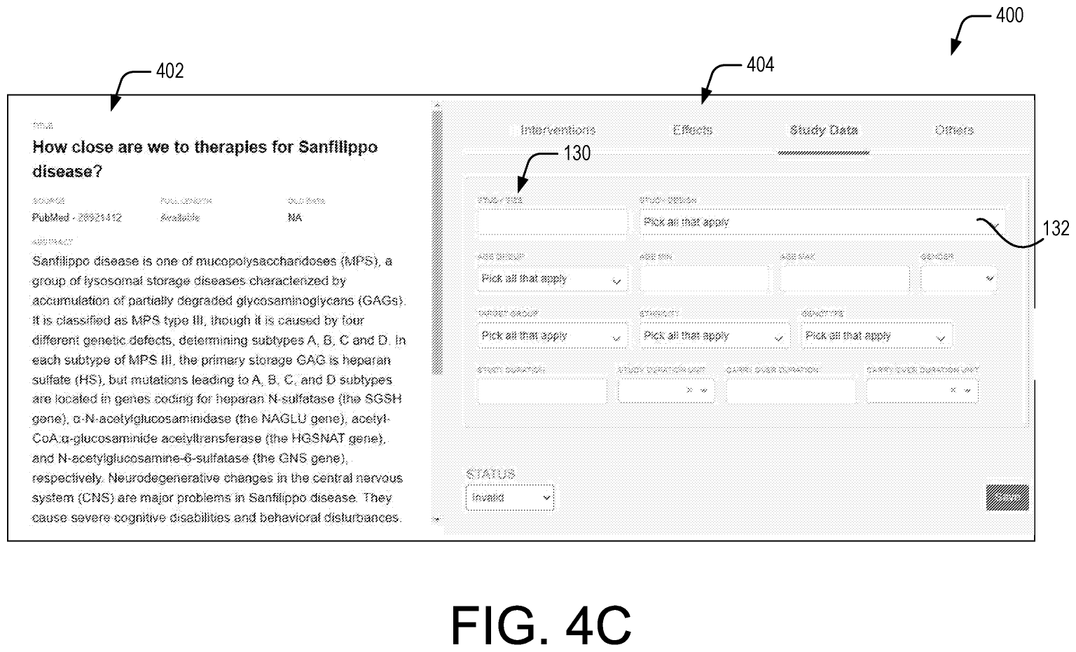

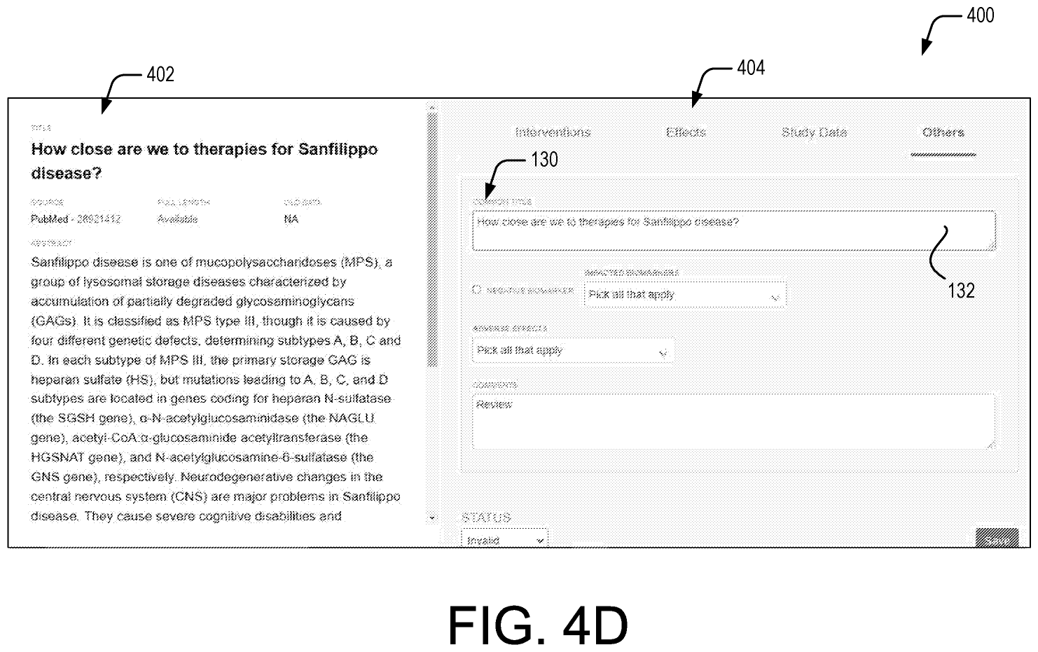

For example, tag value(s) 132 for each document characteristic fields 130 are entered by the user by selecting one of the values provided in a drop-down menu. It may be noted that, for each document characteristic fields 130, the drop down menu contains only those tag value(s) 132 which are present in the selected document and in case of additional tag value(s) 132, these may be added manually by the user as an additional value for future references. Once all document characteristic fields 130 got their corresponding tag value(s) 132, these tags value(s) 132 are associated with the selected document to obtain a tagged document 134. It may be noted that the associated tag values may have different types of formats for different document characteristic fields 130. For example, age, study size or dosage related fields have numerical tag value and gender, ingredient or health condition related fields may have alphabetical word-based tag value. In one example, FIG. 4A-4D depicts the manner in which individually selected document is displayed with corresponding document characteristic fields for tagging. It may be noted that the example of the document characteristic fields 130 as depicted in FIG. 4A-4D is only illustrative, and should not be construed to limit the scope of the present subject matter. Any other document 108 and corresponding document characteristic fields 130 may also be included in the database repository 104 without deviating from the scope of the present subject matter.

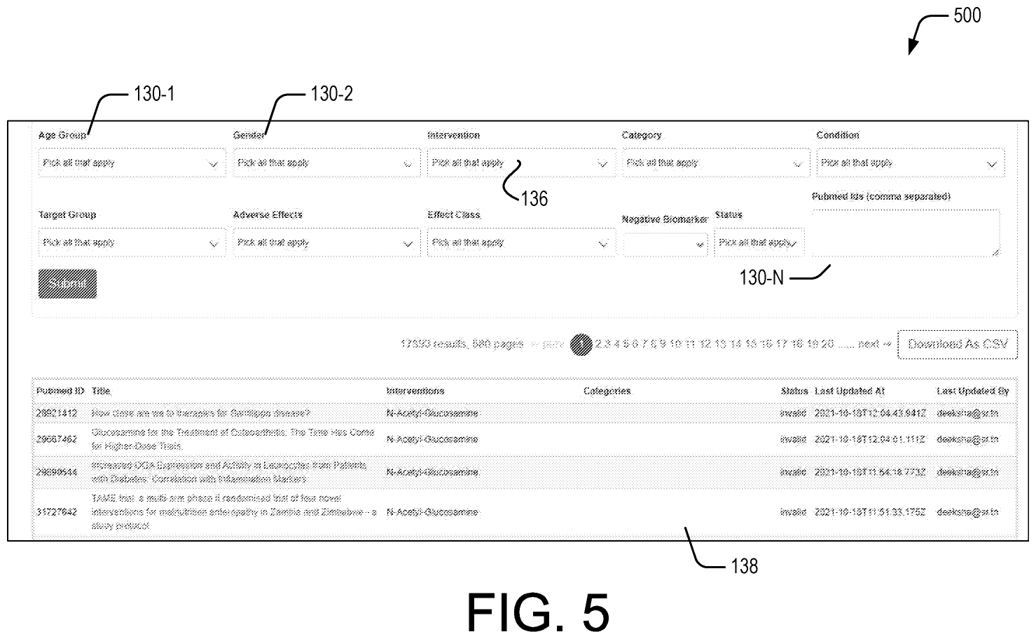

As may be understood, in the manner described above, a large number of documents may be tagged by one or more users, and stored in the same or different database repository. Once a large number of such tagged documents 134 are stored in the database repository 104, this repository may be utilised by random user, such as wellness provider, or medical practitioner, for searching and retrieving document of their need by specifying a request, such as a search request, on the system 102. The search request specifies certain target values 136 for document characteristic fields which may or may not match to the tag values of corresponding document characteristics fields of the tagged documents 134. In one example, FIG. 5 depicts the manner in which a search screen facilitates searching of the tagged documents by entering target values in corresponding document characteristic fields.

The system 102 may then compare the target values 136 with the tag value(s) 132 associated with one or more document characteristic fields 130 corresponding to each tagged document in the database repository 104. Thereafter, based on the result of comparison, a list of tagged document 138 is retrieved or extracted from the database repository 104. In an example, the extracted list of tagged documents 138 comprises documents similar or near similar to the to be searched target document. Once retrieved, the list of tagged documents 138 is displayed for reviewing by the user.

In an example, the ordering of the documents in the extracted list of tagged documents 138 may depends on the number of correct matching between the tag values 132 and target values 136 of corresponding document characteristic fields 130. For example, higher the number of correct matches between the tag values 132 and the target values 136 of the document, higher will be the position of the document in the extracted list of tagged documents 138.

FIG. 2 illustrates an exemplary view of document extracted from the database repository displayed in a listed manner, in accordance with one implementation of the present subject matter. As depicted in FIG. 2, the extracted documents 126 are categorized under several categories, namely, total document 202, new documents 204, draft documents 206, under review documents 208, and published documents 210. In an example, the documents categorized under several categories are further segregated based on the intervention used in the document. For example, the total documents 202 are further categorized under the name of vitamin-D, fish oil, vitamin-C, etc.

FIG. 3 illustrates an exemplary view of documents initially searched based on a document related attributes for individually selecting the document, in accordance with one implementation of the present subject matter. Once the extracted documents 126 are displayed in categorized manner, the system 102 enables the user to further narrow down its search by searching documents based on the document related attributes 128. In an example, the document related attributes 128 includes, but may not be limited to, document repository ID 128-1, supplementary intervention 128-2 used in the document, health benefit 128-3, study title 128-4, status 128-5, etc. Now, the documents searched based on the document related attributes 128 may be selected individually by selected one of the documents using editing option 302 to enable the tagging of the document.

FIG. 4A-4D illustrates an exemplary view of an individually selected document with corresponding document characteristic fields for tagging, in accordance with one implementation of the present subject matter. The data corresponding to individually selected document 400 is displayed in two sections, namely, left section 402 and right section 404. In left section 402, the bibliographic data of selected document 400 is shown. The bibliographic data includes, but may not be limited to, title, source of the document, availability status of full length, abstract, etc. Further, the right section 404 of the selected document 400 shows plurality of document characteristic fields 130 pertaining to the content of the selected document 400. For each document characteristic fields 130, the tag values 132 is provided by the data scientist after performing contextual analysis of the selected document 400.

FIG. 5 illustrates an exemplary view of a search screen facilitating searching of the tagged documents by entering target values in corresponding document characteristic fields, in accordance with one implementation of the present subject matter. As depicted in FIG. 5, a search screen 500 is displayed for receiving or obtaining a search request from the user. The search request comprises target values 136 entered for each of the document characteristic fields 130-1, 130-2, . . . , 130-N. In an example, based on the requirement, the user provides target values 136 for each of the document characteristic fields 130 and the searching module 122 uses the entered target values 136 to identify list of tagged documents 138. As shown in FIG. 5, the documents identified based on the inputted search request are displayed in a listed form as list of tagged documents 138.

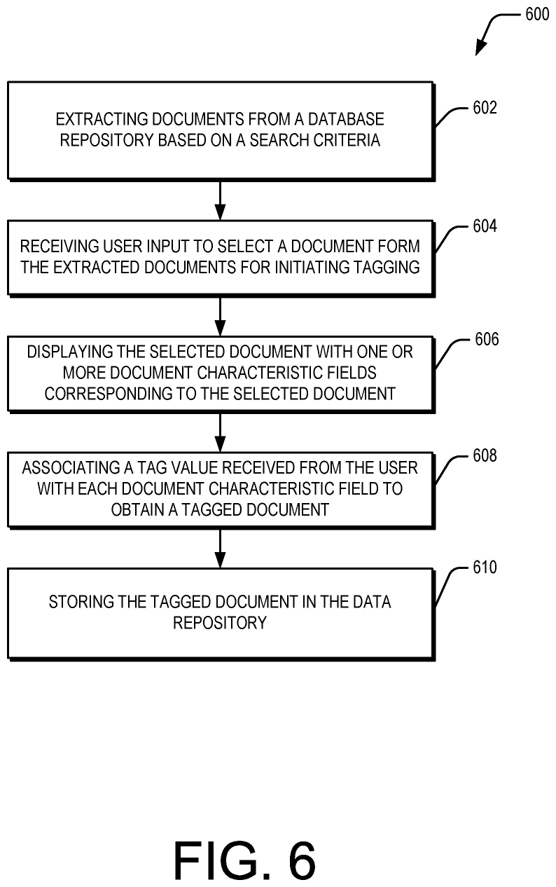

FIG. 6 illustrates a method 600 to be implemented by system 102, as per an example of the present subject matter. The blocks of the method 600 may be implemented through instructions stored in a non-transitory computer-readable medium, as will be readily understood. The non-transitory computer-readable medium may include, for example, digital memories, magnetic storage media, such as magnetic disks and magnetic tapes, hard drives, or optically readable digital data storage media.

At block 602, documents stored in the document repository are retrieved or extracted by the system for tagging. For example, the system 102 may extracts documents 108 from the database repository 104 based on a search query. In an example, retrieved or extracted documents are stored as extracted document 126 in the data 118. In one example, the system 102 may be wirelessly couple to the database repository 104 over the communication network 106 through which the system 102 extracts documents 108 from the database repository 104. In an example, the database repository 104 corresponds to any open source database which contains large number of documents 108 related to the clinical trial studies. In one example, the manner in which various documents 108 retrieved from the repository 104 may be displayed in the system as depicted in FIG. 2. It may be note that the extracted documents 126 are segregated in different columns, such as total documents, new documents, draft documents, under review documents, and published documents. In an example, these extracted documents 126 may also be segregated based on the document related attributes 128 while stored in the document repository, these document related attributes 128 may also be utilised for initial searching of the document for tagging. Examples of such document related attributes 128 includes, but are not limited to, supplementary intervention, document repository ID, health condition and health benefit.

At block 604, an individual document is selected for tagging from the retrieved document. For example, a user input may be received by the tagging module 120 to select the individual document related to the clinical trial for analysing its content by reading, and accordingly tagging the document with specific tag value(s) 132. In an example, once the extracted documents 126 are populated in the system 102, these studies or documents are displayed by tagging module 120 for user to select individual document.

At block 606, document characteristics fields related to the context or concept of the selected document are displayed for user's input. For example, on selecting an individual document, the tagging module 120 displays a plurality of document characteristic fields 130 for inputting tag value(s) 132 for corresponding field. Examples of such document characteristic fields 130 include, but are not limited to, health categories, health condition, intervention, co-supplement used, co-morbidities, target group, ethnicity, genotype, formulation, dosage, frequency, duration, study type, clinical trial rating system, study size, age group, gender, effects, efficacy ratings, adverse effects, negative biomarker effect, etc. It may be noted that additional document characteristic fields 130 may be added for further categorization of the document. In an example, a user analyses the selected document by reading and then manually associating tag value(s) 132 based on the assessment to each document characteristic fields 130.

At block 608, a tag value received from the user is associated with corresponding document characteristic field to obtain a tagged document. For example, once the user analyses the selected document based on its context or concept, a tag value 132 is provided by the user. In an example, tag value(s) 132 for each document characteristic fields 130 are entered by the user by selecting one of the values provided in a drop-down menu. It may be noted that, for each document characteristic fields 130, the drop-down menu contains only those tag value(s) 132 which are present in the selected document and in case of additional tag value(s) 132, these may be added manually by the user as an additional value. It may be noted that the tag value(s) 132 may have different types of values for different fields. For example, age, study size or dosage related fields have numerical tag value and gender, ingredient or health condition related fields may have alphabetical word-based tag value.

Once the tag values are received from the user, the tagging module 120 associates the tag values 132 with corresponding document characteristic fields 130 to obtain the tagged document 134. For example, the tag value(s) 132 corresponding to document characteristic fields 130 are associated by the tagging module 120 to obtain the tagged document 134.

At block 610, the tagged document is stored in the database repository. For example, once the tagging of the selected document is completed, the tagged document 134 is stored in the database repository 104. The tagged documents 134 stored in the tagged data repository may be searched and retrieved by user based on the associated tag values(s) 132 for medication purposes. It may be noted that, the tagged documents 134 may also be stored in a tagged database repository other than the database repository 104, without deviating from the scope of the present subject matter.

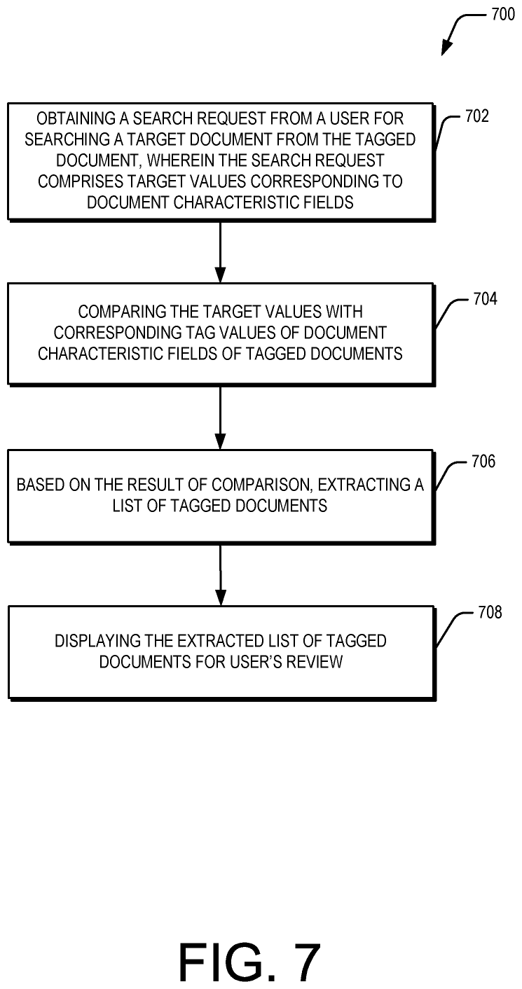

FIG. 7 illustrates a method 700 for searching or retrieving document stored in a tagged data repository, as per an example of the present subject matter. The blocks of the method 700 may be implemented through instructions stored in a non-transitory computer-readable medium, as will be readily understood. The non-transitory computer-readable medium may include, for example, digital memories, magnetic storage media, such as magnetic disks and magnetic tapes, hard drives, or optically readable digital data storage media.

At block 702, a search request from a user for searching a target document is received. For example, the searching module 122 of system 102 may receive a search request specifying certain target values 136 for document characteristic fields 130 for searching the target document. The target values 136 of the document characteristic fields 130 are those values based on which the user wanted to search the documents. In an exemplary case, the searching module 122 may display some fields for receiving user input regarding target values. In one example, FIG. 5 depicts the manner in which a search screen facilitates searching of the tagged documents by entering target values in corresponding document characteristic fields.

In another example, the searching of target document is accomplished in a layered manner. For example, the system 102 may display, on receiving search query, document characteristic fields 130 on basis of which the tagged documents are broadly segregated in the database repository 104. In one example, these broadest categories may be health goal, ingredient used, age group, gender, etc. Thereafter, the system 102 may display subsequent document characteristic fields 130 for specifically selecting the required document.

At block 704, the data extraction system may compare the target values with the tag values tagged to one or more document characteristic fields corresponding to each tagged document stored in the data repository. For example, the searching module 122 compare the target values 136 specified in the search request with the tag value(s) 132 tagged to one or more document characteristic fields 130 corresponding to each document stored in the database repository 104. In another example, the searching module 122 may search documents from the database repository 104 based on the profile data of the user stored in the system 102. In an example, the profile data includes characteristic fields similar to the document characteristic fields 130. Based on the values corresponding to each characteristic field, searching module 122 retrieves documents relevant to the particular user.

At block 706, a list of documents is extracted or retrieved based on the result of comparison from the data repository. For example, the searching module 122 based on the result of comparison, extract the list of tagged documents 138 form the database repository 104. In another example, retrieved documents may be listed based on the target values of some of the document characteristic fields. For example, in FIG. 5, the list of documents is displayed based on the received value for no document characteristics fields. As the user enters the target values, the list changes based on the result of comparison. It may be noted that the list shown in FIG. 5 is only indicative. Other examples may also be provided without limiting the scope of the present subject matter. Thereafter, the user is able to see documents related to the individual health goal or other document characteristic fields by selecting one of the health goals shown in FIG. 5. In this manner, the documents categorically stored in the tagged data repository are retrieved based on the tag values provided by the user.

At block 708, the retrieved list of documents is displayed for user's review. For example, the searching module 122 displays these retrieved documents in listed form. In an example, the ordering of the documents in the extracted list of tagged documents 138 may depends on the number of correct matching between the tag values 132 and target values 136 of corresponding document characteristic fields 130. For example, higher the number of correct matches between the tag values 132 and the target values 136 of the document, higher will be the position of the document in the extracted list of tagged documents 138.

Although examples for the present disclosure have been described in language specific to structural features and/or methods, it is to be understood that the appended claims are not necessarily limited to the specific features or methods described. Rather, the specific features and methods are disclosed and explained as examples of the present disclosure.

1. A data analysis system comprising:
a processor; and
a tagging module coupled to the processor,
wherein the tagging module is to:
extract documents from a database repository based on a search query received from a user;
receive user input to select a document from the extracted document for initiating tagging;
cause the selected document to be displayed with one or more document characteristic fields, wherein the document characteristic fields comprises a set of predefined fields pertaining to a content of the selected document;
associate a tag value received from the user with corresponding document characteristic field to obtain a tagged document, wherein the tag value is a value identified for the corresponding document characteristic filed by analysing the content of the selected document; and
store the tagged document in the database repository.

2. The data analysis system as claimed in claim 1, wherein the documents comprised in the database repository is a clinical trial document, medical research study document, and research paper.

3. The data analysis system as claimed in claim 2, wherein with the selected document related to clinical trials, the search query comprises information pertaining to type of document, type of section of the document, and species on which the document is based.

4. The data analysis system as claimed in claim 3, wherein with the selected document related to clinical trials, the document characteristic fields comprises health categories, health condition, intervention, co-supplement used, co-morbidities, target group, ethnicity, genotype, formulation, dosage, frequency, duration, study type, clinical trial rating system, study size, age group, gender, effects, efficacy ratings, adverse effects, and negative biomarker effect.

5. The data analysis system as claimed in claim 1, wherein the tag values entered for each document characteristic field is selected from one of the values provided as a drop-down menu or by manually inputting the tag values.

6. The data analysis system as claimed in claim 4, wherein the document characteristic fields further comprise sub field which correspond to a set of tag values from amongst the plurality of the tag value entered by the user for corresponding document characteristic field.

7. The data analysis system as claimed in claim 1, wherein the tagging of the documents is performed manually by analysing the content of the documents.

8. The data analysis system as claimed in claim 1, wherein with the database repository comprising tagged documents, the system further comprises a searching module for searching documents from the tagged documents, wherein the searching module is to:
compare a target value with a tag value associated with the corresponding document characteristic field, wherein the target value is derived front a search request, received from the user for searching a target document;
based on the result of comparison, extracting a list of tagged documents comprising target document.

9. A method comprising:
obtaining a search request from a user for searching a target document, wherein the search request comprises target values corresponding to document characteristic fields;
comparing the target values with corresponding tag values of the document characteristic fields pertaining to the tagged document;
based on the result of comparison, extracting a list of tagged documents, wherein the extracted list of tagged documents comprises documents similar or near similar to the to be searched target document; and
causing the extracted list of tagged documents to be displayed for user's review.

10. The method as claimed in claim 9, wherein the ordering of the documents in the extracted list of tagged documents depends on the number of correct matching between the tag values with the target values of the document characteristic fields.

11. The method as claimed in claim 10, wherein higher the number of correct matches between the tag values and the target values of the document, higher the position of the document in the extracted list of tagged documents.

12. The method as claimed in claim 9, wherein with the document related to the clinical trial, the document characteristic fields comprises health categories, health condition, intervention, co-supplement used, co-morbidities, target group, ethnicity, genotype, formulation, dosage, frequency, duration, study type, clinical trial rating system, study size, age group, gender, effects, efficacy ratings, adverse effects, and negative biomarker effect.

13. The method as claimed in claim 9, wherein the tagged documents are retrieved from a database repository.

14. The method as claimed in claim 9, wherein the tagging of the documents is performed manually by analysing content of the documents.

15. The method as claimed in claim 9, wherein the target values and tag values are one of an alphanumerical values, numerical values, and textual values based on the type of document characteristic field.
